(19) United States
(12) Patent Application Publication
(10) Pub. No.: US 2025/0040805 A1
(43) Pub. Date: Feb. 6, 2025

(54) EYEWEAR EQUIPPED WITH PUPIL DIAMETER MEASUREMENT FUNCTION

(71) Applicant: NATSUME RESEARCH INSTITUTE, CO., LTD., Tokyo (JP)

(72) Inventors: Tadashi USUKURA, Tokyo (JP); Hachiro YUGE, Tokyo (JP)

(73) Assignee: NATSUME RESEARCH INSTITUTE, CO., LTD., Tokyo (JP)

(21) Appl. No.: 18/719,251

(22) PCT Filed: Dec. 15, 2022
(86) PCT No.: PCT/JP2022/046276

§ 371 Date: Jun. 13, 2024

(30) Foreign Application Priority Data
Dec. 16, 2021 (JP) 2021-204666

(51) Int. Cl. A61B 3/113; A61B 3/00; A61B 3/14; A61B 90/50

(52) U.S. Cl. CPC A61B 3/113; A61B 3/0025; A61B 3/005; A61B 3/14; A61B 2090/502

(57) ABSTRACT

[Problem] To provide an eyewear capable of appropriately measuring a pupil diameter in consideration of a screen brightness. [Solution] This eyewear for pupil diameter measurement has a display screen and is for measuring the pupil diameter of a target person, the eyewear having: an image brightness acquisition part for acquiring a screen brightness which is information pertaining to the brightness of a display screen; an imaging part for acquiring a target person image which includes the pupil of the target person; and a pupil diameter calculation part for using the screen brightness and the target person image to obtain the pupil diameter of the target person after brightness correction.

### TECHNICAL FIELD

The present invention relates to eyewear equipped with a pupil diameter measurement function.

### BACKGROUND ART

Japanese Utility Model Registration No. 3137375 describes a pupil imaging device.

The pupil imaging device can observe light response of pupils to light stimulation in light-shielding goggles. However, since the device uses light-shielding goggles, the brightness of light other than the light stimulation in the light-shielding goggles is not considered. In addition, due to the use of the light-shielding goggles, the device is not suited to eyewear other than light-shielding goggles.

### CITATION LIST

### Patent Literature

Patent Literature 1: Japanese Utility Model Registration No. 3137375

### SUMMARY OF INVENTION

### Technical Problem

In view of this, the present invention is directed to providing eyewear that is capable of appropriately measuring the pupil diameter in consideration of screen brightness.

### Solution to Problem

An invention described herein is based on the finding that a screen brightness acquisition unit for obtaining screen brightness is included to determine the pupil diameter of a subject after brightness correction.

The present invention relates to pupil diameter measurement eyewear 1.

The pupil diameter measurement eyewear 1 is a tool that has a display screen 3 and is for measuring the pupil diameter of a subject.

The pupil diameter measurement eyewear 1 includes a screen brightness acquisition unit 5 for obtaining screen brightness that is information on the luminosity of the display screen 3, an imaging unit 7 that obtains a subject image that is an image of the subject's pupils, and a pupil diameter calculation unit 9 that determines the pupil diameter of the subject after brightness correction using the screen brightness and the subject image.

The pupil diameter measurement eyewear 1 may be a head mounted display (preferably, a head mounted display for cross reality), a helmet, goggles, eyeglasses, or sunglasses.

A preferred example of the pupil diameter measurement eyewear 1 further includes a line-of-sight position acquisition unit 11 that obtains a line-of-sight position of the subject on the display screen 3, and an attention part analysis unit 13 that determines a subject's attention part in the display image displayed on the display screen 3, using the line-of-sight position acquired by the line-of-sight position acquisition unit 11 and the pupil diameter of the subject after brightness correction.

In this example, the pupil diameter measurement eyewear 1 may further include a display information acquisition unit 15 that acquires analyzer display information for displaying the display screen 3 in an analyzer display part.

An example of the analyzer display information may be the subject's attention part in the display image displayed on the display screen 3.

Another example of the analyzer display information is information for displaying the line-of-sight position acquired and analyzed by the line-of-sight position analysis unit on the display screen 3 displayed in the analyzer display part and for changing the color, size, or shape of the line-of-sight position displayed in the analyzer display part, based on the pupil diameter of the subject calculated by the pupil diameter calculation unit.

### Advantageous Effects of Invention

According to the present invention, it is possible to provide eyewear that is capable of appropriately measuring the pupil diameter in consideration of screen brightness.

### BRIEF DESCRIPTION OF DRAWINGS

FIG. 1 is an example of a block diagram of pupil diameter measurement eyewear according to the present invention.

FIGS. 2A and 2B are graphs as substitutes for diagrams illustrating examples of measurement of the pupil diameter of a subject while playing a commercial moving image on a display screen. FIGS. 2A and 2B are graphs that show changes in the pupil diameter of the subject where a commercial highly interesting for the subject and a commercial less interesting for the subject are played, respectively.

FIGS. 3A to 3C are graphs as substitutes for diagrams illustrating examples of medical diagnoses using pupil reaction. FIG. 3A is a graph of a 73-year-old healthy man, FIG. 3B is a graph of a 59-year-old man with giddiness (Meniere's disease), and FIG. 3C is a graph of a 76-year-old man with Parkinson's disease.

FIG. 4 is a diagram illustrating a display example of an analyzer display part.

FIG. 5 is a graph that indicates temporal changes of pupil diameter of a subject who perceives contents.

FIG. 6 is a diagram representing graph data of temporal transition of a subject's attention degree and average values in output file data. The lateral axis of the graph in FIG. 6 is a time axis, and the vertical axis indicates the degree of the subject's attention.

FIG. 7 is a diagram illustrating total distribution data of a subject's attention degree in output file data.

### DESCRIPTION OF EMBODIMENTS

Hereinafter, embodiments for carrying out the present invention will be described with reference to the drawings. The present invention is not limited to the following embodiments and includes modifications of the following embodiments that might be made by a person skilled in the art as appropriate to the obvious extent.

The pupil diameter measurement eyewear 1 described below may basically have hardware including a control unit, a calculation unit, a storage unit, an input unit, and an output unit. The control unit causes the calculation unit to execute predetermined calculation processing in accordance with a program stored in the storage unit based on information input from the input unit, stores the results of the calculation processing in the storage unit, and controls the output unit. Examples of the control unit and the calculation unit include processors such as a CPU and a GPU. The storage function of the storage unit can be implemented by a non-volatile memory such as an HDD or an SSD, for example. The memory function of the storage unit can be implemented by a volatile memory such as a RAM or a DRAM. Examples of the input device include a communication module for receiving information via a network and operation modules such as a mouse and a keyboard. Examples of the output device includes a communication module for transmitting information via a network, a display, a speaker, and others.

For example, the storage unit may store a control program and may store various kinds of information. When predetermined information is input from the input device, the control unit reads the control program from the storage unit. The control unit then reads the information from the storage unit as appropriate and transfers the same to the calculation unit. The control unit also transfers input information as appropriate to the calculation unit. The calculation unit performs calculation processing using the received various kinds of information and stores the calculation results in the storage unit. The control unit reads the calculation results from the storage unit and outputs the same from the output unit. In this manner, various processes are executed. These various processes are executed by the corresponding units.

### Pupil Diameter Measurement Eyewear 1

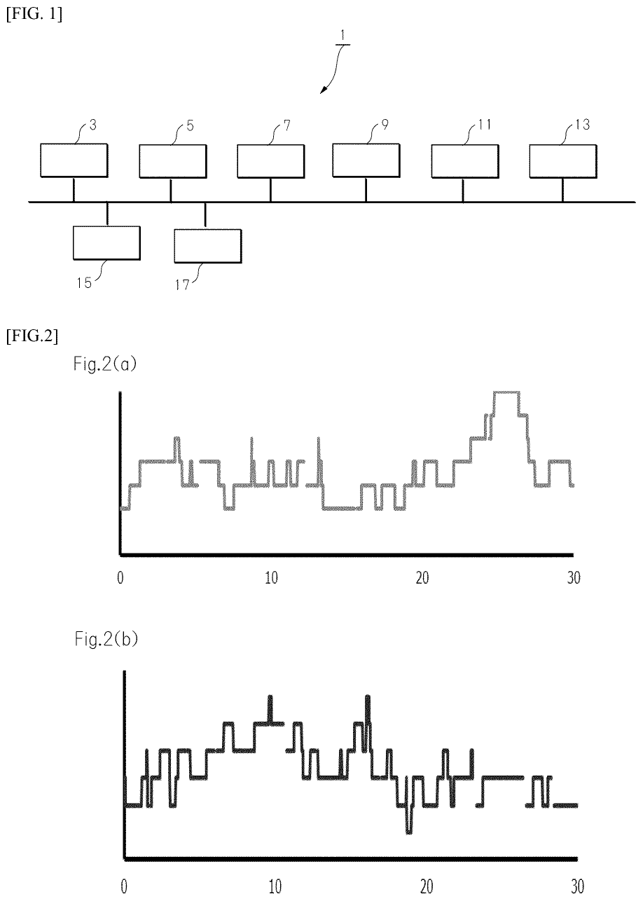

FIG. 1 is an example of a block diagram of pupil diameter measurement eyewear according to the present invention.

As illustrated in FIG. 1, the pupil diameter measurement eyewear 1 includes a display screen 3, a screen brightness acquisition unit 5, an imaging unit 7, and a pupil diameter calculation unit 9. As illustrated in FIG. 1, the pupil diameter measurement eyewear 1 may further include a line-of-sight position acquisition unit 11, an attention part analysis unit 13, and a display information acquisition unit 15. The obtained pupil diameter is used for various applications, for example, such as evaluation of moving images (for example, commercials), questionnaire survey, the evaluation of product display, the evaluation of architectural designs, psychological analysis in playing games, the analysis of sentiment on displayed information, the detection of situation of autonomic nerve, the detection of situation of dementia, the examination of brain disorder, the analysis of cerebral status, and psychoanalysis.

The pupil diameter measurement eyewear 1 is a tool that has the display screen 3 and is for measuring the pupil diameter of a subject. The subject is generally a person. The pupil diameter is generally the pupil diameter of the person.

The main body of the pupil diameter measurement eyewear 1 is capable of measuring the pupil, attachable to the human body, and portable. Examples of the main body of the pupil diameter measurement eyewear 1 include a head mounted display (preferably, head mounted display for cross reality), a helmet, goggles, eyeglasses, and sunglasses. Examples of cross reality (XR) include virtual reality (VR), augmented reality (AR), and mixed reality (MR). The pupil diameter measurement eyewear 1 may be intended for playing a game, working, displaying television programs or video images, or making a virtual tour.

### Display Screen 3

The pupil diameter measurement eyewear 1 has the display screen 3. The display screen 3 is a display part on a liquid crystal display or a plasma display. The display screen shows various kinds of information in response to instructions from the control unit. Examples of contents displayed on the display screen include moving images and still images.

### Screen Brightness Acquisition Unit 5

The screen brightness acquisition unit 5 is an element for obtaining screen brightness that is information on the luminosity of the display screen 3. If the pupil diameter measurement eyewear 1 is a light-shielding object such as light-shielding goggles, the screen brightness may be obtained based on the display information on the display screen 3. Also in this case, the screen brightness of the screen brightness 3 may be determined using a sensor.

### Imaging Unit 7

The imaging unit 7 is an element for obtaining a subject image that is an image of the subject's pupils. Examples of the imaging unit 7 include a camera in the pupil diameter measurement eyewear 1. The imaging unit 7 such as a camera can capture a subject image. The subject image may be a still image or a moving image. Japanese Utility Model Registration No. 3137375 describes an example of the imaging unit 7. In a preferred mode of the present invention, the imaging unit 7 has a near-infrared irradiation device that irradiates the subject (an area including his/her eyes) with near infrared rays in order to easily acquire the subject image. In this case, the camera may be a near-infrared camera.

### Pupil Diameter Calculation Unit 9

The pupil diameter calculation unit 9 is an element for determining the pupil diameter of the subject after brightness correction, using the screen brightness and the subject image. Japanese Patent No. 6651536 describes an example of a method for determining the pupil diameter from a camera image. The determined pupil diameter of the subject after brightness correction is stored as appropriate in the storage unit. At this time, the pupil diameter is preferably stored in the storage unit together with the identification number of the contents (video image) displayed on the display unit. This makes it possible to store the information with the timing of displaying the contents and the timing of changes in the pupil diameter in association with each other.

With the above-described configuration, the pupil diameter measurement eyewear 1 can obtain the pupil diameter of a subject after brightness correction in consideration of the screen brightness. The psychological status of the subject can be grasped by inspecting temporal changes in the pupil diameter. For example, a mechanical learning unit 17 is trained to learn information on the subject to be examined. Then, information on the obtained pupil diameter of the subject after brightness correction is input to the mechanical learning unit. Accordingly, the mechanical learning unit performs various target evaluations and diagnoses based on (temporal changes in) the input pupil diameter of the subject after brightness correction. At this time, if personal information of the subject (age, gender, previous diseases, etc.) is input, the mechanical learning unit can perform evaluations and diagnoses with higher exactitude.

FIGS. 2A and 2B are graphs as substitutes for diagrams illustrating examples of measurement of the pupil diameter of a subject while playing a commercial moving image on the display screen. FIGS. 2A and 2B are graphs that show changes in the pupil diameter of the subject where a commercial highly interesting for the subject and a commercial less interesting for the subject are played, respectively. The vertical axes indicate the pupil diameter (arbitrary unit) and the lateral axes indicate the playing time (seconds). It can be understood from FIGS. 2A and 2B that the subject's interest can be analyzed by the use of the pupil diameter measurement eyewear 1.

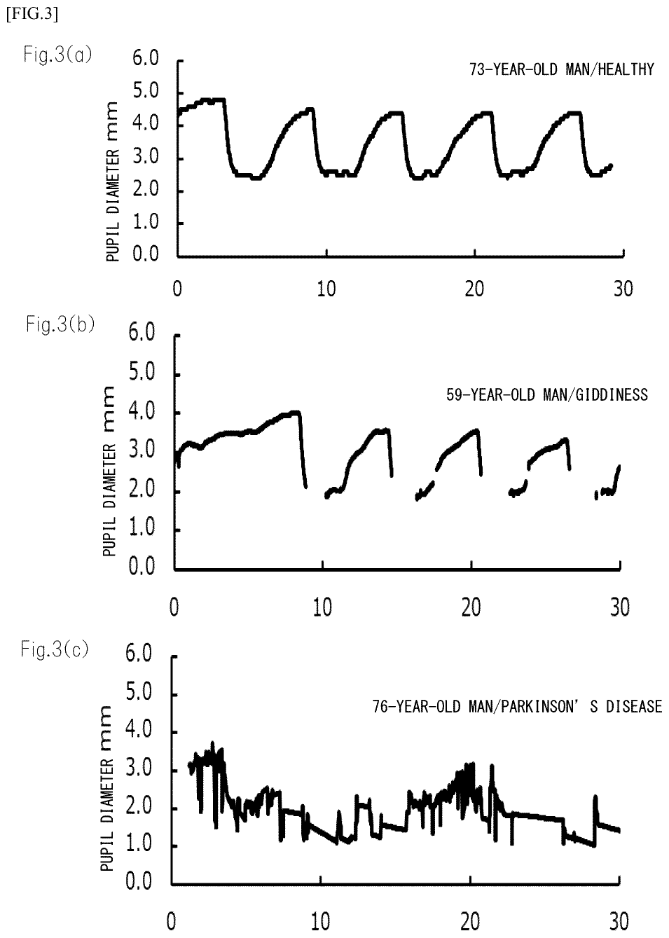

FIGS. 3A to 3C are graphs as substitutes for diagrams illustrating examples of medical diagnoses using pupil reaction. FIG. 3A is a graph of a 73-year-old healthy man, FIG. 3B is a graph of a 59-year-old man with giddiness, and FIG. 3C is a graph of a 76-year-old man with Parkinson's disease. The vertical axes indicate the pupil diameter (arbitrary unit) and the lateral axes indicate the playing time (seconds). When the obtained information on the pupil diameter of a subject after brightness correction is input to the mechanical learning unit 17, the mechanical learning unit 17 analyzes the subject's disease and psychological status based on the input information. It can be understood from FIGS. 3A to 3C that the subject can be medically analyzed by the use of the pupil diameter measurement eyewear 1.

### Line-of-Sight Position Acquisition Unit 11

The line-of-sight position acquisition unit 11 is an element for obtaining the line-of-sight position of a subject on the display screen 3. The line-of-sight position acquisition unit 11 analyzes the point-of-view position of the subject from an image captured by the imaging unit, a video image displayed on the display screen, and information on the eye movement of the subject. The method for analyzing the point-of-view position of the subject is publicly known. The line-of-sight position acquisition unit 11 acquires an eye movement video image captured by the imaging unit. Next, if the contents perceived by the subject is a video image information of a moving image, a still image, or the like, the line-of-sight position acquisition unit 11 acquires the video image information as the video image information of the contents. The line-of-sight position acquisition unit 11 then determines the position in the video image visually recognized by the subject from the video image information of the contents and the eye movement video image, and saves the determined information in the storage unit. Outputting the information stored in the storage unit makes it possible to show the part of the contents (video image) displayed on the display screen that has been visually recognized by the subject. This information can be used for various analyses.

### Attention Part Analysis Unit 13

The attention part analysis unit 13 is an element for determining the part in the display image on the display screen 3 that attracts the subject's attention, using the line-of-sight position acquired by the line-of-sight position acquisition unit 11 and the pupil diameter of the subject after brightness correction. It can be said that as the pupil diameter becomes large, the degree of attention increases. The attention part analysis unit 13 can show how the degree of the subject's attention has changed together with the part in the contents (video image) that has been visually recognized by the subject. The attention part analysis unit 13 can be used to determine on which part in the video image contents the subject perceiving the contents has focused attention. For example, the attention part analysis unit 13 receives the information on the video image contents perceived by the subject, the information on the eye movement of the subject, and others, in the same manner as with the line-of-sight position analysis unit 7 described above. Then, the attention part analysis unit 13 stores the information on the part in the video image screen and the degree of subject's attention on the part in the storage unit. Using these kinds of information, it is possible to add the subject's line-of-sight position represented by various markers described below, to the pupil analysis result moving image displayed on an analyzer display part 4.

### Display Information Acquisition Unit 15

The display information acquisition unit 15 may further has a display information acquisition unit 15 that acquires analyzer display information for displaying the display screen 3 on the analyzer display part. The analyzer display part is a display part to be seen by a person who performs various analyses of the subject. The analyzer display part may be the same as the display screen or may be another display part (for example, the monitor of a computer). For example, the information analyzed by the attention part analysis unit 13 and stored in the storage unit is analyzer display information. The display information acquisition unit 15 outputs the analyzer display information to the analyzer's computer. Accordingly, the video image based on the analyzer display information is displayed on the analyzer display part. As above, an example of the analyzer display information may be the subject's attention part in the display image on the display screen 3.

Another example of the analyzer display information is information on the line-of-sight position acquired and analyzed by the line-of-sight position analysis unit, which is displayed on the display screen 3 on the analyzer display part and is used by the pupil diameter calculation unit to change the color, size, or shape of the line-of-sight position displayed on the analyzer display part, based on the calculated pupil diameter of the subject. This makes it possible to display the part attracting the subject's attention in an easy-to-understand manner.

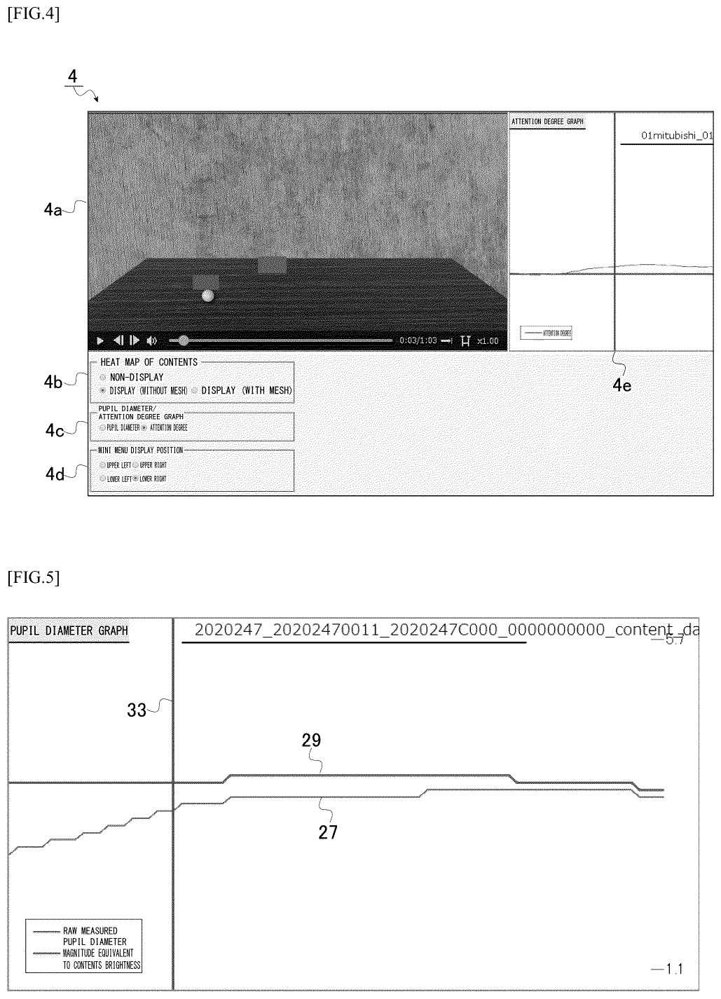

FIG. 4 is a diagram illustrating a display example on the analyzer display part. FIG. 4 is a conceptual diagram illustrating a display example on the analyzer display part. The analyzer display part 4 is an element for displaying the result of pupil analysis of the subject perceiving contents. The analyzer display part 4 may operate on the web or may operate offline in a terminal such as a personal computer. As illustrated in FIG. 4, the analyzer display part may have an analysis result moving image display field 4a for displaying the degree of the subject's attention and the result of the pupil analysis, for example.

The analyzer display part may be provided with a contents heat map setting field 4d, a pupil diameter/attention degree graph display setting field 4c, a mini menu display position setting field 4d, and a pupil diameter/attention degree graph display field 4e.

The analysis result moving image display field 4a is a field where a video image of contents is displayed in which the line-of-sight position and the attention degree of the subject perceiving the contents are associated with each other. Specifically, for example, the video image displayed in the analysis result moving image display field 4a is obtained by adding information on the line-of-sight position and the attention part to video image information of the contents perceived by the subject, as described below. The line-of-sight position of the subject perceiving the contents is additionally displayed as a predetermined heat map marker on the basic video image.

That is, the actual line-of-sight position of the subject perceiving the contents and the position of the heat map marker in the video image information correspond to each other. The marker may be changed in color, size, or shape in accordance with the degree of the subject's attention.

Under the analysis result moving image display field 4a, a moving image replay button, a rewind button, and a fast-forward button may be provided, for example. Besides, a mini menu for setting the replay position and the replay speed may be displayed in the analysis result moving image display field 4a.

The analyzer display part may include the heat map setting field 4b such as a radio box in which the user (analyzer) can select whether to display the heat map marker or the like in the analysis result moving image. For example, if the user selects the non-display of the heat map setting field 4b and presses the replay button, the moving image is replayed in the state where no marker of the attention part is displayed in the analysis result moving image. If the user selects the display of the contents heat map setting field and presses the replay button, the moving image is replayed in the state where the marker of the attention part is displayed in the analysis result moving image. Further, in the case of displaying the marker in the analysis result moving image, mesh lines may be displayed in the moving image.

The analyzer display part may include the pupil diameter/attention degree graph selection field 4c that has a radio box with which the user can select which of the graphs described above is to be displayed.

If the user selects the radio box of the pupil diameter, the pupil diameter graph is displayed in the pupil diameter/attention degree graph display field 4e. If the user selects the radio box of the attention degree, the attention degree graph is displayed in the pupil diameter/attention degree graph display field 4e.

The analyzer display part may have the pupil diameter/attention degree graph display field 4d in which the pupil diameter graph indicating the pupil diameter of the subject and the attention degree graph indicating the degree of the subject's attention are displayed. Either one of the graph indicating the pupil diameter of the subject and the graph indicating the degree of the subject's attention may be displayed or both of the graphs may be displayed.

Besides, the analyzer display part may include the mini menu display position setting field 4d. In the mini menu display position setting field 4d, the mini menu display position can be set.

The graph in FIG. 5 is a graph indicating temporal changes in the pupil diameter of a subject during perception of contents. The lateral direction of the graph indicates the degree of temporal separation from the temporal position in the analysis result moving image during replaying (replayed moving image temporal position line 33).

In the graph, the replayed moving image temporal position line 33 along the vertical line indicates the replaying position (time) in the pupil analysis result moving image displayed on the display screen 3. Provided on the left of the replayed moving image temporal position line 33 in the graph is information on the attention degree at the replayed time position on the display screen 3. Provided on the right of the same is information on the attention degree at the not-yet-replayed time position on the display screen 3.

The vertical direction of the graph (the upward direction in FIG. 5 is the normal direction) indicates the pupil diameter and the magnitude equivalent to the contents brightness.

In the graph, a lateral line of a raw measured pupil diameter 27 is a line formed by a set of points indicating the pupil diameter of the subject at each replaying time position. A lateral line of magnitude equivalent to contents brightness 29 in the graph is a line formed by a set of points indicating the pupil diameter of the subject that is assumed from the contents brightness at each replaying time position. That is, as the replaying position (time) of the analysis moving image advances, the lines of the raw measured pupil diameter 27 and the magnitude equivalent to contents brightness 29 shift to the left side of the graph. The specification of the pupil diameter graph may not be as described above but may be as described below. In another specification, the raw measured pupil diameter 27 and the magnitude equivalent to contents brightness 29 for all the times of the analysis result moving image may be fixedly displayed prior to the replay of the moving image, and the replayed moving image temporal position line 33 may shift in corresponding with the position (time) during the replay of the moving image.

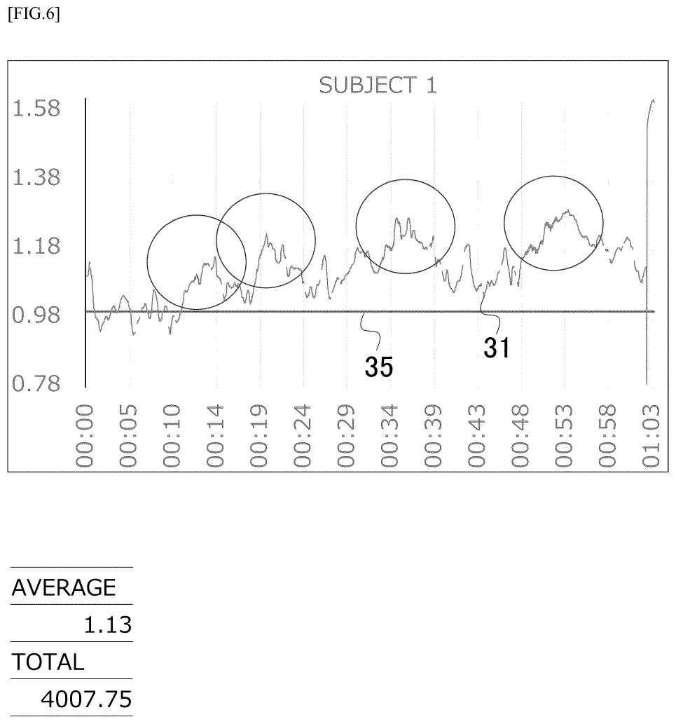

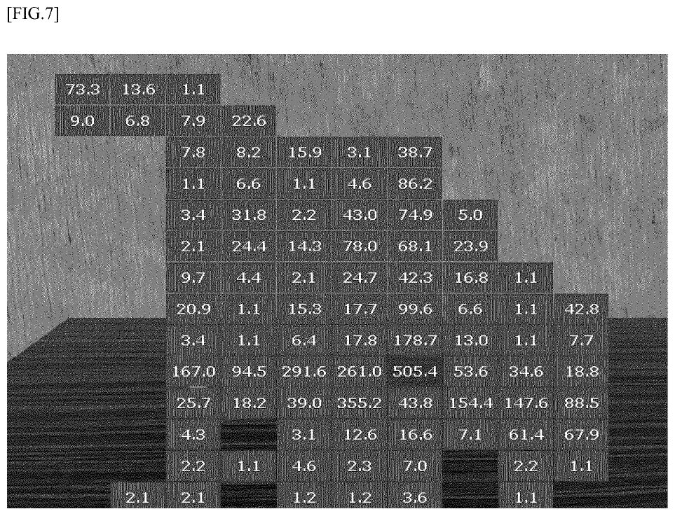

FIG. 6 is a diagram representing graph data of temporal transition of a subject's attention degree and average values in output file data. The lateral axis of the graph in FIG. 6 is a time axis, and the vertical axis indicates the degree of the subject's attention. FIG. 7 is a diagram illustrating total distribution data of a subject's attention degree in output file data. The total attention degree distribution data is aggregated data of the total distributions of the subject's attention degree in the output file data. Besides the above-described files, a file including information on standard deviations indicating variations in the attention degree among a plurality of subjects and a file including information on standard deviations indicating variations in the attention degree of one subject during measurement may be output.

As above, embodiments of the present invention have been described with reference to the drawings in order to implement the present invention. However, the present invention is not limited to the above embodiments, and includes obvious modifications and improvements made by a person skilled in the art, based on the matters described herein.

### INDUSTRIAL APPLICABILITY

The present invention can be utilized in the field of information analysis.

### REFERENCE SIGNS LIST

1 Pupil diameter measurement eyewear
3 Display screen
5 Screen brightness acquisition unit
7 Imaging unit
9 Pupil diameter calculation unit
11 Line-of-sight position acquisition unit
13 Attention part analysis unit
15 Display information acquisition unit

1. Pupil diameter measurement eyewear that has a display screen and is for measuring a pupil diameter of a subject, comprising:
a screen brightness acquisition unit for obtaining screen brightness that is information on luminosity of the display screen;
an imaging unit that obtains a subject image that is an image of the subject's pupils; and
a pupil diameter calculation unit that determines the pupil diameter of the subject after brightness correction using the screen brightness and the subject image.

2. The pupil diameter measurement eyewear according to claim 1, wherein the pupil diameter measurement eyewear is a head mounted display.

3. The pupil diameter measurement eyewear according to claim 1, wherein the pupil diameter measurement eyewear is a head mounted display for cross reality.

4. The pupil diameter measurement eyewear according to claim 1, wherein the pupil diameter measurement eyewear is a helmet, goggles, eyeglasses, or sunglasses.

5. The pupil diameter measurement eyewear according to claim 1, further comprising:
a line-of-sight position acquisition unit that obtains a line-of-sight position of the subject on the display screen; and
an attention part analysis unit that determines a subject's attention part in the display image displayed on the display screen, using the line-of-sight position acquired by the line-of-sight position acquisition unit and the pupil diameter of the subject after brightness correction.

6. The pupil diameter measurement eyewear according to claim 5, further comprising
a display information acquisition unit that acquires analyzer display information for displaying the display screen in an analyzer display part, wherein
the analyzer display information is information for displaying the line-of-sight position acquired and analyzed by the line-of-sight position analysis unit on the display screen displayed in the analyzer display part and for changing color, size, or shape of the line-of-sight position displayed in the analyzer display part, based on the pupil diameter of the subject calculated by the pupil diameter calculation unit.
